(19) United States
(12) Patent Application Publication
(10) Pub. No.: US 2018/0000416 A1
(43) Pub. Date: Jan. 4, 2018

(54) GARMENT-BASED ERGONOMIC ASSESSMENT

(71) Applicant: Pawankumar Hegde, Folsom, CA (US)

(72) Inventor: Pawankumar Hegde, Folsom, CA (US)

(21) Appl. No.: 15/200,400

(22) Filed: Jul. 1, 2016

(51) Int. Cl. A61B 5/00; A41D 1/04; A61B 5/11; A41D 13/02; A61B 5/22; A41D 1/00

(52) U.S. Cl. CPC A61B 5/6805; A61B 5/221; A61B 5/746; A61B 5/7275; A61B 5/7267; A61B 5/224; A41D 1/002; A61B 5/1123; A61B 5/1116; A41D 13/02; A41D 1/04; A61B 2503/20

(57) ABSTRACT

Embodiments are directed to ergonomic monitoring. A plurality of diverse motion-assessment sensors are incorporated into a garment. Output from groups of diverse sensors associated with corresponding regions of the garment is obtained. Predefined movements by a wearer of the garment are recognized. The predefined movements are associated with respective parameters that represent characteristics of the movements. The values of the parameters are determined, and an ergonomic impact of any of the recognized movements is assessed based on the recognized movements, the parameter values, and predefined ergonomic impact criteria.

### TECHNICAL FIELD

Embodiments described herein generally relate to health and wellness and, more particularly, to apparatus and associated methods for performing automated ergonomic assessment.

### BACKGROUND

Of all workplace injuries, the majority are attributable to ergonomic problems. These may include improper posture, over-exertion, repetitive motion, and the like. Traditionally, ergonomic concerns were addressed by having specialist health-care providers observe the work routines of employees, and provide education, training, and suggestions for improving work area layout, equipment-human interfaces, and the like. Although the conventional approach has been beneficial, it is impractical to have specialists at a workplace to observe every employee over time. Unfortunately, having only periodic (at best) or merely occasional ergonomics consultations leaves employees mostly un-observed, opening the door for them to revert to their ergonomically-problematic posture and work habits. Accordingly, there is a need for a practical solution that addresses the challenges of ergonomic assessment.

### BRIEF DESCRIPTION OF THE DRAWINGS

In the drawings, which are not necessarily drawn to scale, like numerals may describe similar components in different views. Like numerals having different letter suffixes may represent different instances of similar components. Some embodiments are illustrated by way of example, and not limitation, in the figures of the accompanying drawings.

FIG. 1 is a high-level system diagram illustrating an example arrangement of a system for monitoring ergonomics according to some embodiments.

FIG. 2 is a block diagram illustrating components of an example controller, and an example deep analyzer according to some embodiments.

FIG. 3 is a block diagram illustrating an example system architecture of a processor-based computing device according to an embodiment.

FIG. 4 is a diagram illustrating an exemplary hardware and software architecture of a computing device such as the one depicted in FIG. 3, in which various interfaces between hardware components and software components are shown.

FIG. 5 is a block diagram illustrating examples of processing devices that may be implemented on a computer system, such as the computer system described with reference to FIGS. 3-4, according to some embodiments.

FIG. 6 is a block diagram illustrating example components of a CPU as one of the processing devices depicted in FIG. 5, according to various embodiments.

FIG. 7 is a diagram illustrating an example architecture of controller that may be incorporated with a garment according to some embodiments.

FIG. 8 is a flow diagram illustrating operations performed by a controller, optionally with a server, according to various embodiments.

### DETAILED DESCRIPTION

Some aspects of the embodiments are directed to automated monitoring of ergonomics using a plurality of diverse garment-mounted motion-assessment sensors. The posture and motion of a wearer of the garment is monitored. Although posture and motion are somewhat different concepts, with posture generally relating to static positioning and motion generally relating to dynamic translational or rotational movement, in the present context, the term “motion” shall refer to posture or motion, unless a specific distinction is expressly made.

Advantageously, the use of garment-mounted sensors in some embodiments greatly simplifies engagement and disengagement with the sensors for the wearer of the garment, and helps with repeatability of sensor placement. In certain work environments, such as cleanroom facilities, laboratories, factories, and the like, workers tend to wear specialized garments for avoidance of contamination, safety, protection of clothing from dust or chemicals, worker visibility, etc. Accordingly, the addition of sensors to such garments tends to be unobtrusive for the wearer.

The sensors are monitored by a specialized system, at least a portion of which may be implemented using a computing platform through the execution of program instructions thereupon. The computing platform may be one physical machine, or may be distributed among multiple physical machines, such as by role or function, or by process thread in the case of a cloud computing distributed model. In various embodiments certain operations may run in virtual machines that in turn are executed on one or more physical machines. It will be understood by persons of skill in the art that features of the embodiments may be realized by a variety of different suitable machine implementations.

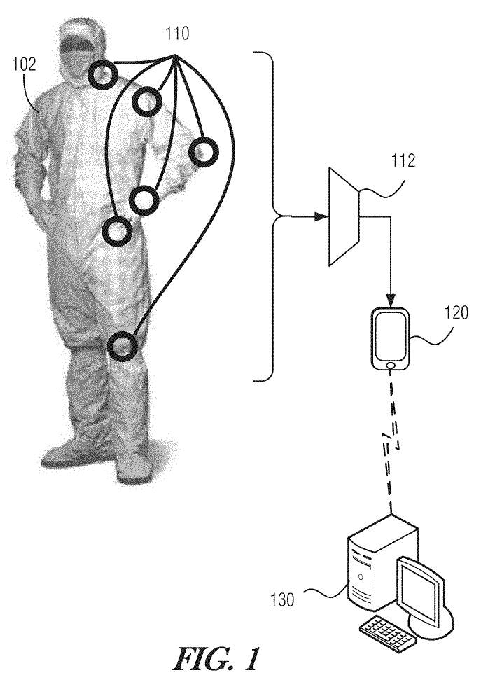

FIG. 1 is a high-level system diagram illustrating an example arrangement of a system for monitoring ergonomics according to some embodiments. Garment 102 includes a plurality of motion-assessment sensors 110. A variety of garment types are contemplated according to various embodiments. For instance, a cleanroom suit, as depicted, is typically used in cleanrooms within semiconductor, spacecraft, precision instrument, pharmaceutical, or sterile device fabrication plants. The example suit as shown covers the entire region of the body and has the potential for sensor placement for data collection across the many regions of the body.

In another example (not shown), a lab smock is used as the garment 102 on which the plurality of sensors are incorporated. Lab smocks are used within labs where the entire body need not be covered unlike the cleanroom scenario. Typically, a lab smock is worn with a pair of the gloves. As a further example of garment 102, vests are used in maintenance and construction sites where lifting of heavy items is commonplace.

Various types of sensors 110 are contemplated according to embodiments. The sensors may include inertial motion sensing (e.g., accelerometer, gyroscopic, magnetometer, etc.) The sensors may include strain gauges, load cells, and various biometric sensing technologies, such as heart rate, temperature, electromyography (EMG) sensing to detect exertion of the wearer's musculature. Also, surface moisture sensing may be used to detect perspiration of the wearer. In addition, environmental indicia may be measured, such as particulate matter (PM) using a PM sensor.

Placement of the sensors is generally selected to be ergonomically relevant. In various embodiments, as depicted, sensors 110 may be placed at joints, such as the neck, shoulder, elbow, forearm, wrist, hip, knee, ankle, etc. Also, sensors 110 may be placed in the upper and lower back regions.

In an embodiment, wrist placement of a sensor 110 is on the posterior side of the palm in a configuration to detect flexion, extension and ulnar deviation, from which an accurate strain index value for wrist may be computationally obtained. In a related embodiment, one or more sensors 110 may be placed in a detachable glove with options of completely covering the fingertips or leaving them partially opened.

In an embodiment, shoulder placement is on the sleeve's frontal side at mid-arm region. Using a snug fitted flap may provide an accurate description of shoulder orientations such as flexion, extension and abduction, from which an accurate strain index value for may be obtained for the shoulder.

Placement of the neck sensor may be below the collar of the garment where the garment is in direct contact with cervical spine region of the wearer. Placement of a sensor may also be directly on the collar bone. Measured movements include postures of neck-lateral bending, extension, and rotation.

In an embodiment for sensors to measure the back, a sensor may be placed on a back brace and snugly fitted inside the garment through belt loops for an adjustable fit. For example, using this sensor arrangement, bending postures of the wearer's back can be used to calculate risk orders for the National Institute for Occupational Safety and Health (NIOSH) lifting equation.

Notably, in some embodiments, sensors at a particular location or region may include more than one sensing technology. For example, an accelerometer may be used in conjunction with a strain sensor and an EMG sensor. In a related embodiment, the diverse sensors may be integrated in a common package or housing.

In another related embodiment, diverse sensors 110 of different locations or regions are analyzed together to infer a type of movement or gesture by the wearer. For instance, concurrent motion of the knees, back, shoulders, elbows, and head, may be indicative of the wearer bending down to pick up a large or heavy object. These indicia may distinguish a wearer's activity from a different activity of squatting down, for instance, which may be inferred when only a single region's sensors are analyzed in isolation from other regions.

Data from sensors 110 is interfaced with controller 120, which is constructed, programmed, or otherwise configured, to read sensors 110 and associated with various regions of the garment, recognize movements made by the wearer of the garment, and assess an ergonomic impact of any of the recognized movements, according to an embodiment.

The recognizable movements may be predefined in controller 120, and associated with respective parameters that represent characteristics of those movements. The controller 120 may also determine values of the parameters, and make its assessment of the ergonomic impact of any of the recognized movements based on the recognized movements, the parameter values, and predefined ergonomic impact criteria.

The interface between sensors 110 and controller 120 may include analog-to-digital conversion (ADC), including such operations as sampling, quantizing, and encoding the sensor data, and multiplexing the data from individual sensors at interface hardware 112.

In the embodiment depicted, controller 120 is communicatively coupled with remote server 130, which hosts a deep analyzer that is constructed, programmed, or otherwise configured, to collect movement information over a monitoring period, and to perform task classification of the movement information to assess an ergonomic performance of the wearer.

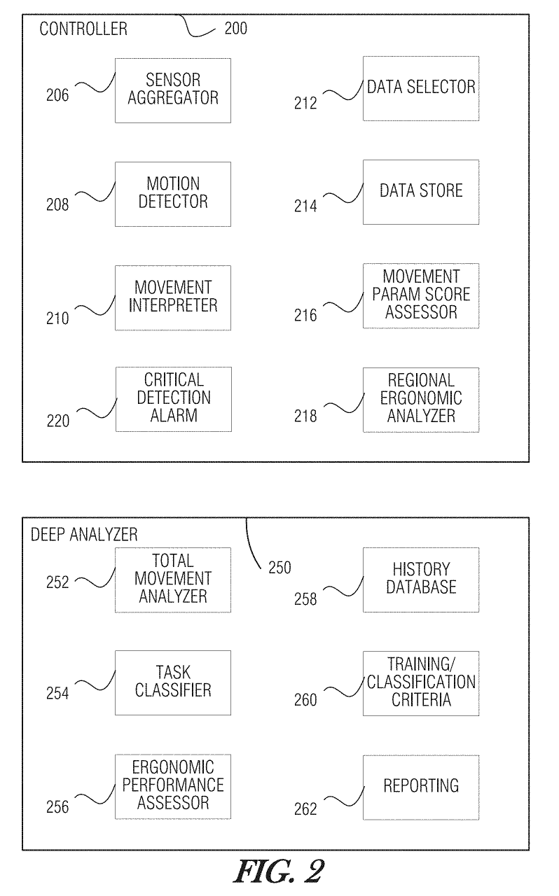

FIG. 2 is a block diagram illustrating components of an example controller 200, and an example deep analyzer 250 according to some embodiments. In various embodiments, these components are implemented as engines, circuits, components, or modules, which for the sake of consistency are termed engines, although it will be understood that these terms may be used interchangeably. Engines may be hardware, software, or firmware communicatively coupled to one or more processors in order to carry out the operations described herein. Engines may be hardware engines, and as such engines may be considered tangible entities capable of performing specified operations and may be configured or arranged in a certain manner. In an example, circuits may be arranged (e.g., internally or with respect to external entities such as other circuits) in a specified manner as an engine. In an example, the whole or part of one or more hardware processors may be configured by firmware or software (e.g., instructions, an application portion, or an application) as a engine that operates to perform specified operations. In an example, the software may reside on a machine-readable medium. In an example, the software, when executed by the underlying hardware of the engine, causes the hardware to perform the specified operations. Accordingly, the term hardware engine is understood to encompass a tangible entity, be that an entity that is physically constructed, specifically configured (e.g., hard wired), or temporarily (e.g., transitorily) configured (e.g., programmed) to operate in a specified manner or to perform part or all of any operation described herein.

Considering examples in which engines are temporarily configured, each of the engines need not be instantiated at any one moment in time. For example, where the engines comprise a general-purpose hardware processor core configured using software; the general-purpose hardware processor core may be configured as respective different engines at different times. Software may accordingly configure a hardware processor core, for example, to constitute a particular engine at one instance of time and to constitute a different engine at a different instance of time.

Sensor aggregator 206 is configured to read multiple individual sensors and obtain output from groups of diverse sensors associated with corresponding regions of the garment. For instance, the output of an accelerometer situated at the left shoulder of the wearer may be grouped with an EMG sensor situated at the left shoulder. Motion detector 208 is configured to analyze the output of the respective sensors to detect signaling indicative of motion. It may apply a thresholding filter to disregard noise. Movement interpreter 210 is configured to recognize predefined movements by a wearer of the garment, such as moving an arm, standing, twisting at the waist, etc. The predefined movements may include flexion, extension, rotation, bending, translation, and the like. The predefined movements are associated with respective parameters that represent characteristics of the movements, such as length of extension, angle of rotation, etc.

Data selector 212 is configured to apply selection criteria to certain data in order to select that data for further analysis. Data store 214 is configured to store some determined amount of raw sensor data, calibrated sensor data, interpreted sensor data, selected data, etc. Movement parameter score assessor 216 is configured to assess the values of the various parameters based on the sensor data such as length of extension, angle of rotation, and the like. Regional ergonomic analyzer 218 is configured to apply ergonomic criteria to the interpreted, parameter-scored movements to ascertain whether any of the movements meet, or are in violation, of predefined ergonomic guidelines. For example, excessive straining, excessive repetition of certain movements within a time window, etc. The predefined ergonomic guidelines may be defined in terms of a strain-index score, for instance. Critical detection alarm 220 is configured to notify the wearer in the event that the ergonomic guidelines are violated. Notification may be via visual, audible, or haptic cues, for example.

Turning to deep analyzer 250, total movement analyzer 252 is configured to collect movement information over a monitoring period. Total movement analyzer 252 may read raw sensor information, calibrated sensor information, or it may read one or more of the outputs of the engines of controller 200, as discussed above, such as movement interpreter 210. Notably, total movement analyzer 252 may examine movement of all regions at once to gather a greater depth of information about the context, and nature, or the movements. In one embodiment, total movement analyzer 252 utilizes a machine-learning process to classify the total movement. Any suitable machine-learning or data-mining process may be utilized, such as K-nearest neighbor classification, clustering, association rule mining, artificial neural network, or the like.

Task classifier 254 is configured to analyze context of the movement, such as whether the movement is a known job or task. For instance, picking and placing items on a conveyor may involve a certain movement pattern that is identifiable as a specific task. Another example of a task, in a lab setting, is bending over to look into a microscope. In a related embodiment, deep analyzer 250 is configured to limit its analysis to only defined task-related movements that are job-related, for instance.

Instances of job behaviors that represent ergonomic risks include standing for long hours, repeated movements or long-duration stretching of the neck, bending of the back, squatting awkwardly, long-duration rotating or repeated bending of the wrist, hunching, lifting and placing tools or other objects repeatedly, looking up at a screen, walking long distances while carrying large objects, repeated movements or long duration slouching of the shoulders, bending to lift the items, lifting, rotating and twisting of an arm in a repetitive pattern.

Ergonomic performance assessor 256 is configured to compare the analyzed movements to ergonomic guideline criteria. The ergonomic guideline criteria used by ergonomic performance assessor 256 may include non-critical guidelines, including best-practices, the violation of which is not considered an immediate health hazard, but that may cause or contribute to a future ergonomic-related problem. For instance, wrist positioning while typing may be a long-term cause for carpal tunnel syndrome, tendonitis, or the like, though by itself it may not be inherently dangerous.

History database 258 stores records of movement data, classification results, ergonomic performance assessments, and the like, in association with a corresponding user account. Training/classification criteria 260 includes training data for total movement analyzer 252, task classifier 254, and ergonomic performance assessor 256. Reporting engine 262 produces reports of ergonomic assessment for the wearer, or the wearer's employer.

In an embodiment, controller 200 periodically passes sensor data, assessments, and other pertinent information to deep analyzer 250 on a near-real-time basis. In another embodiment, data is passed periodically, e.g., according to a schedule. In another embodiment, data is “pulled” by deep analyzer 250 as needed.

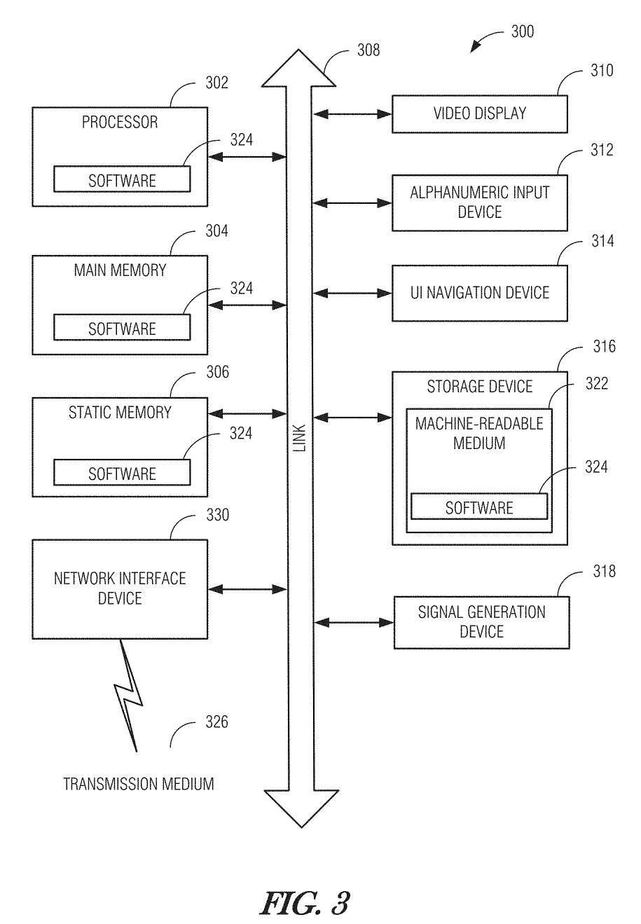

FIG. 3 is a block diagram illustrating a computer system 300 in the example form of a general-purpose machine. In certain embodiments, programming of the computer system 300 according to one or more particular algorithms produces a special-purpose machine upon execution of that programming, to form controller 120, or server 130, for example. In a networked deployment, the computer system 300 may operate in the capacity of either a server or a client machine in server-client network environments, or it may act as a peer machine in peer-to-peer (or distributed) network environments.

Example computer system 300 includes at least one processor 302 (e.g., a central processing unit (CPU), a graphics processing unit (GPU) or both, processor cores, compute nodes, etc.), a main memory 304 and a static memory 306, which communicate with each other via a link 308 (e.g., bus). The computer system 300 may further include a video display unit 310, an alphanumeric input device 312 (e.g., a keyboard), and a user interface (UI) navigation device 314 (e.g., a mouse). In one embodiment, the video display unit 310, input device 312 and UI navigation device 314 are incorporated into a touch screen display. The computer system 300 may additionally include a storage device 316 (e.g., a drive unit), a signal generation device 318 (e.g., a speaker), a network interface device (NID) 320, and one or more sensors (not shown), such as a global positioning system (GPS) sensor, compass, accelerometer, or other sensor.

The storage device 316 includes a machine-readable medium 322 on which is stored one or more sets of data structures and instructions 324 (e.g., software) embodying or utilized by any one or more of the methodologies or functions described herein. The instructions 324 may also reside, completely or at least partially, within the main memory 304, static memory 306, and/or within the processor 302 during execution thereof by the computer system 300, with the main memory 304, static memory 306, and the processor 302 also constituting machine-readable media.

While the machine-readable medium 322 is illustrated in an example embodiment to be a single medium, the term “machine-readable medium” may include a single medium or multiple media (e.g., a centralized or distributed database, and/or associated caches and servers) that store the one or more instructions 324. The term “machine-readable medium” shall also be taken to include any tangible medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

NID 330 according to various embodiments may take any suitable form factor. In one such embodiment, NID 320 is in the form of a network interface card (NIC) that interfaces with processor 302 via link 308. In one example, link 308 includes a PCI Express (PCIe) bus, including a slot into which the NIC form-factor may removably engage. In another embodiment, NID 320 is a network interface circuit laid out on a motherboard together with local link circuitry, processor interface circuitry, other input/output circuitry, memory circuitry, storage device and peripheral controller circuitry, and the like. In another embodiment, NID 320 is a peripheral that interfaces with link 308 via a peripheral input/output port such as a universal serial bus (USB) port. NID 320 transmits and receives data over transmission medium 326, which may be wired or wireless (e.g., radio frequency, infra-red or visible light spectra, etc.), fiber optics, or the like.

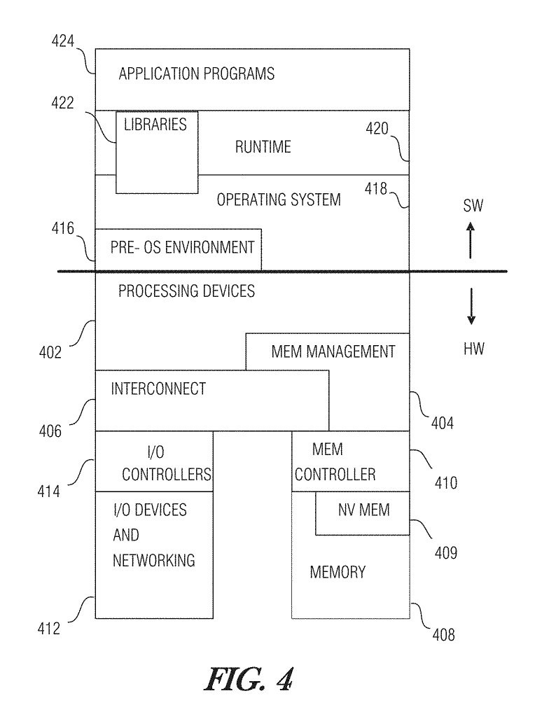

FIG. 4 is a diagram illustrating an exemplary hardware and software architecture of a computing device such as the one depicted in FIG. 3, in which various interfaces between hardware components and software components are shown. As indicated by HW, hardware components are represented below the divider line, whereas software components denoted by SW reside above the divider line. On the hardware side, processing devices 402 (which may include one or more microprocessors, digital signal processors, etc., each having one or more processor cores, are interfaced with memory management device 404 and system interconnect 406. Memory management device 404 provides mappings between virtual memory used by processes being executed, and the physical memory. Memory management device 404 may be an integral part of a central processing unit which also includes the processing devices 402.

Interconnect 406 includes a backplane such as memory, data, and control lines, as well as the interface with input/output devices, e.g., PCI, USB, etc. Memory 408 (e.g., dynamic random access memory—DRAM) and non-volatile memory 409 such as flash memory (e.g., electrically-erasable read-only memory—EEPROM, NAND Flash, NOR Flash, etc.) are interfaced with memory management device 404 and interconnect 406 via memory controller 410. This architecture may support direct memory access (DMA) by peripherals in some embodiments. I/O devices, including video and audio adapters, non-volatile storage, external peripheral links such as USB, Bluetooth, etc., as well as network interface devices such as those communicating via Wi-Fi or LTE-family interfaces, are collectively represented as I/O devices and networking 412, which interface with interconnect 406 via corresponding I/O controllers 414.

On the software side, a pre-operating system (pre-OS) environment 416, which is executed at initial system start-up and is responsible for initiating the boot-up of the operating system. One traditional example of pre-OS environment 416 is a system basic input/output system (BIOS). In present-day systems, a unified extensible firmware interface (UEFI) is implemented. Pre-OS environment 416, is responsible for initiating the launching of the operating system, but also provides an execution environment for embedded applications according to certain aspects of the invention.

Operating system (OS) 418 provides a kernel that controls the hardware devices, manages memory access for programs in memory, coordinates tasks and facilitates multi-tasking, organizes data to be stored, assigns memory space and other resources, loads program binary code into memory, initiates execution of the application program which then interacts with the user and with hardware devices, and detects and responds to various defined interrupts. Also, operating system 418 provides device drivers, and a variety of common services such as those that facilitate interfacing with peripherals and networking, that provide abstraction for application programs so that the applications do not need to be responsible for handling the details of such common operations. Operating system 418 additionally provides a graphical user interface (GUI) that facilitates interaction with the user via peripheral devices such as a monitor, keyboard, mouse, microphone, video camera, touchscreen, and the like.

Runtime system 420 implements portions of an execution model, including such operations as putting parameters onto the stack before a function call, the behavior of disk input/output (I/O), and parallel execution-related behaviors. Runtime system 420 may also perform support services such as type checking, debugging, or code generation and optimization.

Libraries 422 include collections of program functions that provide further abstraction for application programs. These include shared libraries, dynamic linked libraries (DLLs), for example. Libraries 422 may be integral to the operating system 418, runtime system 420, or may be added-on features, or even remotely-hosted. Libraries 422 define an application program interface (API) through which a variety of function calls may be made by application programs 424 to invoke the services provided by the operating system 418. Application programs 424 are those programs that perform useful tasks for users, beyond the tasks performed by lower-level system programs that coordinate the basis operability of the computing device itself.

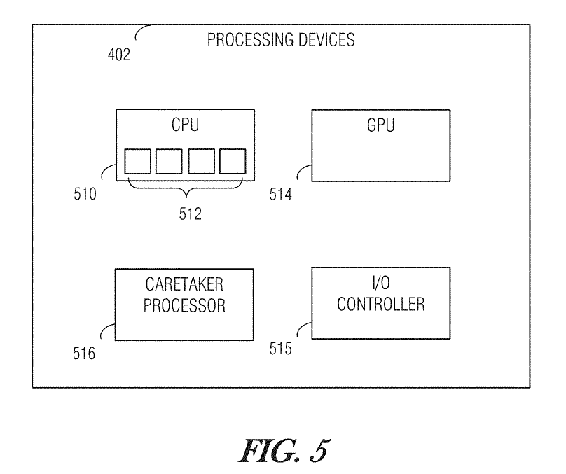

FIG. 5 is a block diagram illustrating processing devices 402 according to some embodiments. In one embodiment, two or more of processing devices 402 depicted are formed on a common semiconductor substrate. CPU 510 may contain one or more processing cores 512, each of which has one or more arithmetic logic units (ALU), instruction fetch unit, instruction decode unit, control unit, registers, data stack pointer, program counter, and other essential components according to the particular architecture of the processor. As an illustrative example, CPU 510 may be an x86-type of processor. Processing devices 402 may also include a graphics processing unit (GPU) 514. In these embodiments. GPU 514 may be a specialized co-processor that offloads certain computationally-intensive operations, particularly those associated with graphics rendering, from CPU 510. Notably, CPU 510 and GPU 514 generally work collaboratively, sharing access to memory resources, I/O channels, etc.

Processing devices 402 may also include caretaker processor 516 in some embodiments. Caretaker processor 516 generally does not participate in the processing work to carry out software code as CPU 510 and GPU 514 do. In some embodiments, caretaker processor 516 does not share memory space with CPU 510 and GPU 514, and is therefore not arranged to execute operating system or application programs. Instead, caretaker processor 516 may execute dedicated firmware that supports the technical workings of CPU 510, GPU 514, and other components of the computer system. In some embodiments, caretaker processor is implemented as a microcontroller device, which may be physically present on the same integrated circuit die as CPU 510, or may be present on a distinct integrated circuit die. Caretaker processor 516 may also include a dedicated set of I/O facilities to enable it to communicate with external entities. In one type of embodiment, caretaker processor 516 is implemented using a manageability engine (ME) or platform security processor (PSP). Input-output (I/O) controller 515 coordinates information flow between the various processing devices 510, 514, 516, as well as with external circuitry, such as a system interconnect.

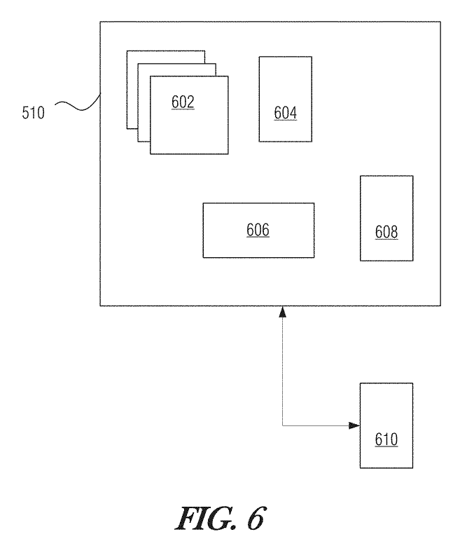

FIG. 6 is a block diagram illustrating example components of CPU 510 according to various embodiments. As depicted, CPU 510 includes one or more cores 602, cache 604, and CPU controller 606, which coordinates interoperation and tasking of the core(s) 602, as well as providing an interface to facilitate data flow between the various internal components of CPU 510, and with external components such as a memory bus or system interconnect. In one embodiment, all of the example components of CPU 510 are formed on a common semiconductor substrate.

CPU 510 includes non-volatile memory 608 (e.g., flash, EEPROM, etc.) for storing certain portions of foundational code, such as an initialization engine, and microcode. Also, CPU 510 may be interfaced with an external (e.g., formed on a separate IC) non-volatile memory device 610 that stores foundational code that is launched by the initialization engine, such as system BIOS or UEFI code.

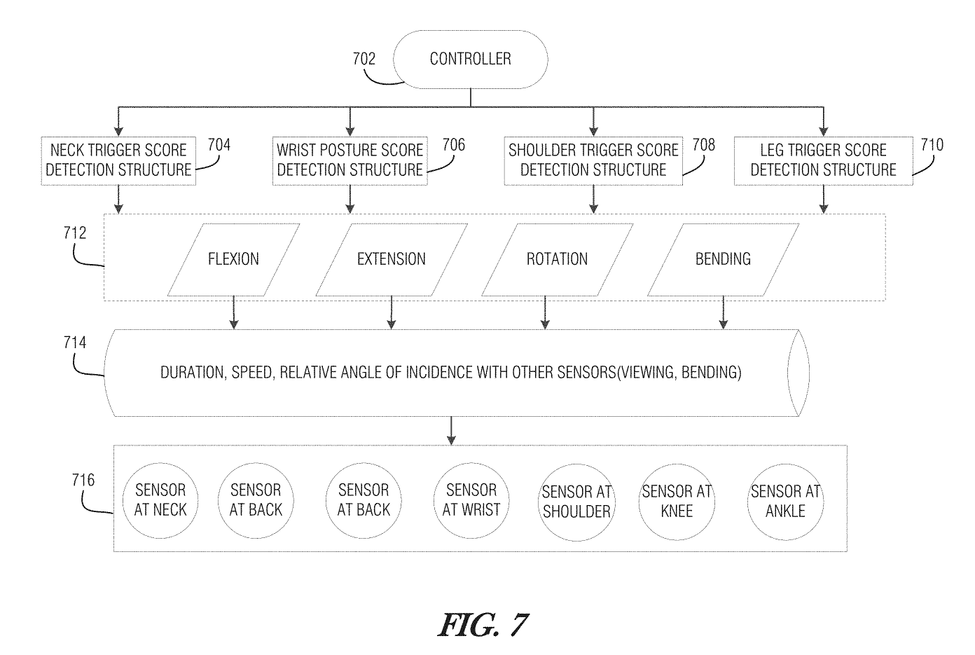

FIG. 7 is a diagram illustrating an example architecture of controller 702 that may be incorporated with a garment 102. Controller 702 includes detection data structures 704-710 that represent criteria for detecting ergonomic guideline violations. These include neck trigger score detection structure 704, wrist posture score detection structure 706, shoulder trigger score detection structure 708, and leg trigger score detection structure 710. Each detection data structure 704-710 may define a type of movement and threshold parameter value(s) that constitute a violation of the ergonomic guidelines.

In order to apply the detection data structures 704-710, movement interpreter 712 is invoked to determine the type, and extent, of movement. Examples of movement, as illustrated, include flexion, extension, rotation, and bending.

Movement is determined by movement interpreter 712 based on motion data analyzer 714. Analyzer 714 reads sensor data from sensors 716 and, knowing the type, location, and output range and sensitivity of each sensor, constructs an assessment of movement. For instance, as illustrated, motion data analyzer 714 assesses measured speed, relative angle of incidence, viewing, bending, etc., and the duration thereof, based on the sensor data.

In a related aspect, some embodiments are directed to logistics of use of a garment with embedded motion-assessment sensors. In one approach, the controller and sensors, where applicable, are battery-powered, necessitating recharging of the battery or batteries. Accordingly, a charging station is implemented in an embodiment using a coat hanger. In an example, the coat hanger contains magnetic blocks at specific locations that enable it to latch with magnetic blocks of garment. In a seamless fashion, when the wearer places the garment onto the coat hanger. The magnetic blocks in the hanger interact with magnetic material incorporated in the garment to retain the garment in a preferred location on the coat hanger, and to establish electrical coupling between a charging circuit and the devices incorporated on the garment.

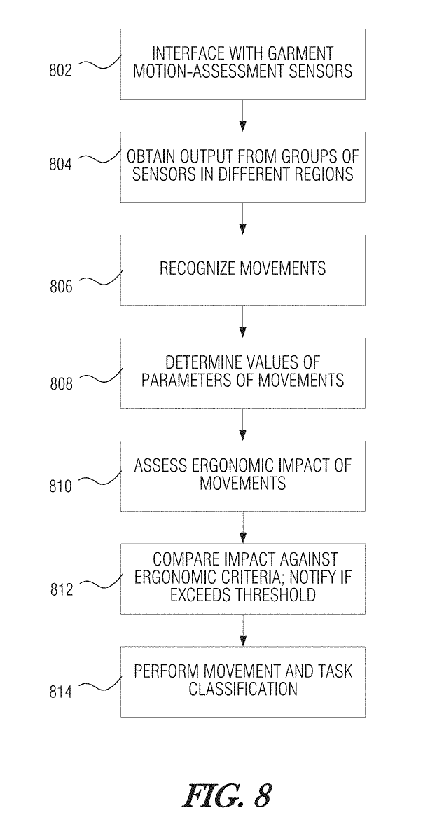

FIG. 8 is a flow diagram illustrating operations performed by controller 120, optionally with server 130, according to various embodiments. It is important to note that the process is a richly-featured embodiment that may be realized as described; in addition, portions of the process may be implemented while others are excluded in various embodiments. The following Additional Notes and Examples section details various combinations, without limitation, that are contemplated. It should also be noted that in various embodiments, certain process operations may be performed in a different ordering than depicted in FIG. 8, provided that the logical flow and integrity of the process is not disrupted in substance.

In FIG. 8, at 802 controller 120 interfaces with the sensors of the garment. At 804, controller 120 obtains output from groups of sensors that correspond to different regions of the garment. At 806, movements are recognized for each region. Each movement is further defined by certain parameters that may vary. In response to the movement recognition, at 808, controller 120 determines parameter values. At 810, the ergonomic impact of the movements is assessed by controller 120. In an example embodiment, safety-related impact is assessed at this stage. In another embodiment, less severe impact may be assessed.

At 812, the assessed ergonomic impact is compared against ergonomic criteria, which may be risk factors, best practices, etc. If the threshold is exceeded, a notification is issued to the wearer of the garment, to the wearer's employer, or both. At 814, movement and task classification is performed by server 130, which may include a machine learning process.

### Additional Notes & Examples

Example 1 is a system for ergonomic monitoring, the system comprising: a controller interfaced with a plurality of diverse motion-assessment sensors incorporated into a garment, the controller including: a sensor aggregator configured to obtain output from groups of diverse sensors associated with corresponding regions of the garment; a movement interpreter configured to recognize predefined movements by a wearer of the garment, the predefined movements being associated with respective parameters that represent characteristics of the movements; a movement parameter score assessor configured to determine values of the parameters; and an ergonomic analyzer configured to assess an ergonomic impact of any of the recognized movements based on the recognized movements, the parameter values, and predefined ergonomic impact criteria.

In Example 2, the subject matter of Example 1 optionally includes wherein the diverse motion-assessment sensors include at least one sensor to measure muscular output of the wearer at specific regions of the body of the wearer.

In Example 3, the subject matter of any one or more of Examples 1-2 optionally include wherein the parameters include posture, velocity, intensity of efforts, and task duration.

In Example 4, the subject matter of any one or more of Examples 1-3 optionally include wherein the garment is a type of garment selected from the group consisting of: a full-body suit, a smock having sleeves and upper-body coverage, a vest, or any combination thereof.

In Example 5, the subject matter of any one or more of Examples 1-4 optionally include wherein the predefined movements are selected from the group consisting of: flexion, extension, rotation, bending, or any combination thereof.

In Example 6, the subject matter of any one or more of Examples 1-5 optionally include an ergonomic notification alarm configured to issue a perceptible notification to the wearer of the garment in response to the ergonomic impact of any of the recognized movements exceeds a safety threshold of the ergonomic impact criteria.

In Example 7, the subject matter of any one or more of Examples 1-6 optionally include a deep analyzer configured to collect movement information over a monitoring period, and to perform movement and task classification of the movement information to assess an ergonomic performance of the wearer.

In Example 8, the subject matter of Example 7 optionally includes wherein the deep analyzer is hosted on a remote server that is communicatively coupled to the controller.

In Example 9, the subject matter of any one or more of Examples 7-8 optionally include wherein the deep analyzer is configured to apply at least one machine learning process to perform the movement and task classification.

In Example 10, the subject matter of any one or more of Examples 7-9 optionally include wherein the deep analyzer is configured to apply a machine learning process to perform the ergonomic performance assessment.

In Example 11, the subject matter of any one or more of Examples 7-10 optionally include wherein the deep analyzer is configured to perform a predictive assessment of an ergonomic risk experienced by the wearer.

In Example 12, the subject matter of any one or more of Examples 1-11 optionally include wherein the plurality of sensors and the controller are coupled to an electrical circuit configured to receive power via a coat hanger.

In Example 13, the subject matter of any one or more of Examples 1-12 optionally include the garment comprising the plurality of diverse motion-assessment sensors.

Example 14 is a machine-implemented method for ergonomic monitoring, the method being carried out by a controller, and comprising: communicatively interfacing with a plurality of diverse motion-assessment sensors incorporated into a garment; obtaining output from groups of diverse sensors associated with corresponding regions of the garment; recognizing predefined movements by a wearer of the garment, the predefined movements being associated with respective parameters that represent characteristics of the movements; determining values of the parameters; and assessing an ergonomic impact of any of the recognized movements based on the recognized movements, the parameter values, and predefined ergonomic impact criteria.

In Example 15, the subject matter of Example 14 optionally includes wherein the diverse motion-assessment sensors include at least one sensor to measure muscular output of the wearer at specific regions of the body of the wearer.

In Example 16, the subject matter of any one or more of Examples 14-15 optionally include wherein the parameters include posture, velocity, intensity of efforts, and task duration.

In Example 17, the subject matter of any one or more of Examples 14-16 optionally include wherein the predefined movements are selected from the group consisting of: flexion, extension, rotation, bending, or any combination thereof.

In Example 18, the subject matter of any one or more of Examples 14-17 optionally include issuing a perceptible notification to the wearer of the garment in response to the ergonomic impact of any of the recognized movements exceeds a safety threshold of the ergonomic impact criteria.

In Example 19, the subject matter of any one or more of Examples 14-18 optionally include collecting movement information over a monitoring period; and performing movement and task classification of the movement information to assess an ergonomic performance of the wearer.

In Example 20, the subject matter of Example 19 optionally includes applying at least one machine learning process to perform the movement and task classification.

In Example 21, the subject matter of any one or more of Examples 19-20 optionally include applying a machine learning process to perform the ergonomic performance assessment.

In Example 22, the subject matter of any one or more of Examples 19-21 optionally include performing a predictive assessment of an ergonomic risk experienced by the wearer.

Example 23 is at least one machine-readable storage medium comprising instructions that, when executed on a computing platform, cause the computing platform to execute the method according to any one of Examples 14-22.

Example 24 is a system for ergonomic monitoring, the system comprising means for executing the method according to any one of Examples 14-22.

Example 25 is a system for ergonomic monitoring, the system comprising: means for communicatively interfacing with a plurality of diverse motion-assessment sensors incorporated into a garment; means for obtaining output from groups of diverse sensors associated with corresponding regions of the garment; means for recognizing predefined movements by a wearer of the garment, the predefined movements being associated with respective parameters that represent characteristics of the movements; means for determining values of the parameters; and means for assessing an ergonomic impact of any of the recognized movements based on the recognized movements, the parameter values, and predefined ergonomic impact criteria.

In Example 26, the subject matter of Example 25 optionally includes wherein the diverse motion-assessment sensors include at least one sensor to measure muscular output of the wearer at specific regions of the body of the wearer.

In Example 27, the subject matter of any one or more of Examples 25-26 optionally include wherein the parameters include posture, velocity, intensity of efforts, and task duration.

In Example 28, the subject matter of any one or more of Examples 25-27 optionally include wherein the predefined movements are selected from the group consisting of: flexion, extension, rotation, bending, or any combination thereof.

In Example 29, the subject matter of any one or more of Examples 25-28 optionally include means for issuing a perceptible notification to the wearer of the garment in response to the ergonomic impact of any of the recognized movements exceeds a safety threshold of the ergonomic impact criteria.

In Example 30, the subject matter of any one or more of Examples 25-29 optionally include means for collecting movement information over a monitoring period; and means for performing movement and task classification of the movement information to assess an ergonomic performance of the wearer.

In Example 31, the subject matter of Example 30 optionally includes means for applying at least one machine learning process to perform the movement and task classification.

In Example 32, the subject matter of any one or more of Examples 30-31 optionally include means for applying a machine learning process to perform the ergonomic performance assessment.

In Example 33, the subject matter of any one or more of Examples 30-32 optionally include means for performing a predictive assessment of an ergonomic risk experienced by the wearer.

In Example 34, the subject matter of any one or more of Examples 25-33 optionally include wherein the plurality of sensors and the controller are coupled to an electrical circuit configured to receive power via a coat hanger.

In Example 35, the subject matter of any one or more of Examples 25-34 optionally include the garment comprising the plurality of diverse motion-assessment sensors.

Example 36 is at least one machine-readable storage medium comprising instructions for ergonomic monitoring, the instructions, when executed by a computing platform, cause the computing platform to: communicatively interface with a plurality of diverse motion-assessment sensors incorporated into a garment; obtain output from groups of diverse sensors associated with corresponding regions of the garment; recognize predefined movements by a wearer of the garment, the predefined movements being associated with respective parameters that represent characteristics of the movements; determine values of the parameters; and assess an ergonomic impact of any of the recognized movements based on the recognized movements, the parameter values, and predefined ergonomic impact criteria.

In Example 37, the subject matter of Example 36 optionally includes wherein the diverse motion-assessment sensors include at least one sensor to measure muscular output of the wearer at specific regions of the body of the wearer.

In Example 38, the subject matter of any one or more of Examples 36-37 optionally include wherein the parameters include posture, velocity, intensity of efforts, and task duration.

In Example 39, the subject matter of any one or more of Examples 36-38 optionally include wherein the predefined movements are selected from the group consisting of: flexion, extension, rotation, bending, or any combination thereof.

In Example 40, the subject matter of any one or more of Examples 36-39 optionally include instructions for: issuing a perceptible notification to the wearer of the garment in response to the ergonomic impact of any of the recognized movements exceeds a safety threshold of the ergonomic impact criteria.

In Example 41, the subject matter of any one or more of Examples 36-40 optionally include instructions for: collecting movement information over a monitoring period; and performing movement and task classification of the movement information to assess an ergonomic performance of the wearer.

In Example 42, the subject matter of Example 41 optionally includes instructions for: applying at least one machine learning process to perform the movement and task classification.

In Example 43, the subject matter of any one or more of Examples 41-42 optionally include instructions for: applying a machine learning process to perform the ergonomic performance assessment.

In Example 44, the subject matter of any one or more of Examples 41-43 optionally include instructions for: performing a predictive assessment of an ergonomic risk experienced by the wearer.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, also contemplated are examples that include the elements shown or described. Moreover, also contemplated are examples using any combination or permutation of those elements shown or described (or one or more aspects thereof), either with respect to a particular example (or one or more aspects thereof), or with respect to other examples (or one or more aspects thereof) shown or described herein.

Publications, patents, and patent documents referred to in this document are incorporated by reference herein in their entirety, as though individually incorporated by reference. In the event of inconsistent usages between this document and those documents so incorporated by reference, the usage in the incorporated reference(s) are supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A.” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third.” etc. are used merely as labels, and are not intended to suggest a numerical order for their objects.

The above description is intended to be illustrative, and not restrictive. For example, the above-described examples (or one or more aspects thereof) may be used in combination with others. Other embodiments may be used, such as by one of ordinary skill in the art upon reviewing the above description. The Abstract is to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. However, the claims may not set forth every feature disclosed herein as embodiments may feature a subset of said features. Further, embodiments may include fewer features than those disclosed in a particular example. Thus, the following claims are hereby incorporated into the Detailed Description, with a claim standing on its own as a separate embodiment. The scope of the embodiments disclosed herein is to be determined with reference to the appended claims, along with the full scope of equivalents to which such claims are entitled.

What is claimed is:

1. A system for ergonomic monitoring, the system comprising:
a controller interfaced with a plurality of diverse motion-assessment sensors incorporated into a garment, the controller including:
a sensor aggregator configured to obtain output from groups of diverse sensors associated with corresponding regions of the garment;
a movement interpreter configured to recognize predefined movements by a wearer of the garment, the predefined movements being associated with respective parameters that represent characteristics of the movements;
a movement parameter score assessor configured to determine values of the parameters; and
an ergonomic analyzer configured to assess an ergonomic impact of any of the recognized movements based on the recognized movements, the parameter values, and predefined ergonomic impact criteria.

2. The system of claim 1, wherein the diverse motion-assessment sensors include at least one sensor to measure muscular output of the wearer at specific regions of the body of the wearer.

3. The system of claim 1, wherein the parameters include posture, velocity, intensity of efforts, and task duration.

4. The system of claim 1, wherein the garment is a type of garment selected from the group consisting of: a full-body suit, a smock having sleeves and upper-body coverage, a vest, or any combination thereof.

5. The system of claim 1, wherein the predefined movements are selected from the group consisting of: flexion, extension, rotation, bending, or any combination thereof.

6. The system of claim 1, further comprising:
an ergonomic notification alarm configured to issue a perceptible notification to the wearer of the garment in response to the ergonomic impact of any of the recognized movements exceeds a safety threshold of the ergonomic impact criteria.

7. The system of claim 1, further comprising:
a deep analyzer configured to collect movement information over a monitoring period, and to perform movement and task classification of the movement information to assess an ergonomic performance of the wearer.

8. The system of claim 7, wherein the deep analyzer is hosted on a remote server that is communicatively coupled to the controller.

9. The system of claim 7, wherein the deep analyzer is configured to apply at least one machine learning process to perform the movement and task classification.

10. The system of claim 7, wherein the deep analyzer is configured to apply a machine learning process to perform the ergonomic performance assessment.

11. The system of claim 7, wherein the deep analyzer is configured to perform a predictive assessment of an ergonomic risk experienced by the wearer.

12. The system of claim 1, wherein the plurality of sensors and the controller are coupled to an electrical circuit configured to receive power via a coat hanger.

13. The system of claim 1, further comprising:
the garment comprising the plurality of diverse motion-assessment sensors.

14. A machine-implemented method for ergonomic monitoring, the method being carried out by a controller, and comprising:
communicatively interfacing with a plurality of diverse motion-assessment sensors incorporated into a garment;
obtaining output from groups of diverse sensors associated with corresponding regions of the garment;
recognizing predefined movements by a wearer of the garment, the predefined movements being associated with respective parameters that represent characteristics of the movements;
determining values of the parameters; and
assessing an ergonomic impact of any of the recognized movements based on the recognized movements, the parameter values, and predefined ergonomic impact criteria.

15. The method of claim 14, wherein the diverse motion-assessment sensors include at least one sensor to measure muscular output of the wearer at specific regions of the body of the wearer.

16. The method of claim 14, wherein the parameters include posture, velocity, intensity of efforts, and task duration.

17. The method of claim 14, further comprising:
issuing a perceptible notification to the wearer of the garment in response to the ergonomic impact of any of the recognized movements exceeds a safety threshold of the ergonomic impact criteria.

18. The method of claim 14, further comprising:
collecting movement information over a monitoring period; and
performing movement and task classification of the movement information to assess an ergonomic performance of the wearer.

19. The method of claim 16, further comprising:
applying at least one machine learning process to perform the movement and task classification.

20. The method of claim 16, further comprising:
applying a machine learning process to perform the ergonomic performance assessment.

21. The method of claim 16, further comprising:
performing a predictive assessment of an ergonomic risk experienced by the wearer.

22. At least one machine-readable storage medium comprising instructions for ergonomic monitoring, the instructions, when executed by a computing platform, cause the computing platform to:
communicatively interface with a plurality of diverse motion-assessment sensors incorporated into a garment;
obtain output from groups of diverse sensors associated with corresponding regions of the garment;
recognize predefined movements by a wearer of the garment, the predefined movements being associated with respective parameters that represent characteristics of the movements;
determine values of the parameters; and
assess an ergonomic impact of any of the recognized movements based on the recognized movements, the parameter values, and predefined ergonomic impact criteria.

23. The at least one machine-readable storage medium of claim 22, wherein the diverse motion-assessment sensors include at least one sensor to measure muscular output of the wearer at specific regions of the body of the wearer.

24. The at least one machine-readable storage medium of claim 22, wherein the parameters include posture, velocity, intensity of efforts, and task duration.

25. The at least one machine-readable storage medium of claim 22, wherein the predefined movements are selected from the group consisting of: flexion, extension, rotation, bending, or any combination thereof.
